(19) United States
(12) Patent Application Publication
(10) Pub. No.: US 2023/0218361 A1
(43) Pub. Date: Jul. 13, 2023

(54) CARRIERS FOR SURGICAL OBJECTS, AND SETS AND CONTAINERS CONTAINING SUCH CARRIERS

(71) Applicant: MEDARTIS HOLDING AG, Basel (CH)

(72) Inventors: Raphael PFEFFERLE, Münstertal (DE); Stefan BRAND, Bern (CH); Philippe Robert KERN, Kleines Wiesental (DE); Marco Andreas MÜLLER, St. Pantaleon (CH); Nikolaus POCHLATKO, Arlesheim (CH); Jürgen SCHONHARDT, Rheinfelden (DE); Seline STEFFANONI, St. Moritz (CH); Hermann ZEUNER, Freiburg (DE)

(21) Appl. No.: 17/799,456

(22) PCT Filed: Feb. 14, 2020
(86) PCT No.: PCT/EP2020/053824

§ 371 Date: Aug. 12, 2022

(51) Int. Cl. A61B 50/33; A61B 50/22

(52) U.S. Cl. CPC A61B 50/33; A61B 50/22; A61B 2050/006

(57) ABSTRACT

The invention relates to supports (1) for receiving a surgical implant, in particular a bone plate (2), containing an implant receptacle (3), the implant receptacle (3) being of substantially elongate form along a longitudinal axis (4) and having a first part (5) and a second part (6), the first part (5) having a shaft (7) for receiving the surgical implant (2) at a recess (13) of the implant (2), the second part (6) having a first fastening portion (8) for fastening to a holding portion (9) of a container (114). The support (1) furthermore contains at least one elastic element (10), in particular a spring, for securing the implant, which is designed to secure an implant, which is held on the implant receptacle (3), by a force component substantially perpendicular to the longitudinal axis (4). Also disclosed are supports (100) for receiving at least one surgical item, in particular an implant, in particular a screw (101).

The present invention is concerned with carriers for holding surgical objects, and specifically, inter alia, implants such as bone plates or bone screws. It is further concerned with sets and containers containing such carriers.

Generic carriers for holding bone plates are disclosed, for example, in WO 2009/024189. The carriers have support portions located in the region of an opening of the bone plate. A support element is made of a thermoplastic material and is elastic. The bone plate is held laterally by the snap elements and pressed towards a base surface. The bone plate is in contact with the retaining elements in such a way that there is no contact between the bone plate and the base surface.

The bone plate holders disclosed in WO 2010/097447 are designed such that they can be stacked on top of each other. The bone plate holders each have an opening into which the bone plates can be inserted laterally.

Although the carriers for bone plates known in the prior art already have a number of advantages, further developments are desirable which simplify the insertion and removal of the bone plate.

Also generic carriers for bone screws are known from the already mentioned document WO 2010/097447. The screw is held on an underside of its head by a support surface of the carrier. In addition, the screw is secured by elastic tongues which rest against the thread of the screw.

Furthermore, document EP 2 392 286 discloses a carrier having a receiving opening into which a screw is inserted. In addition, the carrier has a retaining tongue for securing. In the received position, the retaining tongue secures the screw.

Further developments are also desirable with regard to carriers for bone screws, which, for example, enable even better securing of the screw in the carrier or simpler checking of the securing.

The object thus arises of further developing the carriers known from the prior art for surgical objects, in particular for implants such as bone plates or for bone screws—in particular to the effect that the surgical objects can be inserted and removed more easily and/or that they are better secured against falling out and/or that the securing is easy to check. Hereinafter, securing the surgical object is always understood to mean preventing the surgical object from falling out of the carrier.

In a first aspect, the invention relates to a carrier for receiving a surgical implant, in particular a bone plate. The carrier includes an implant receptacle formed substantially elongate along a longitudinal axis and having a first portion and a second portion. The first portion includes a shaft for receiving the surgical implant at a recess of the implant, and the second portion includes a first attachment portion for attachment to a retaining portion of a container. Optionally, the first attachment portion may also be for positioning on said retaining portion. Furthermore, the carrier includes at least one elastic element, in particular a spring, for securing the implant.

According to the first aspect of the invention, the elastic element is configured to secure, by a component of force substantially perpendicular to the longitudinal axis, an implant held on the implant receptacle. The design of the elastic element according to the invention allows quick securing and easy removal of the implant. In addition, the implant receptacle is suitable for different implant sizes and is therefore flexible in use.

In some embodiments, it is possible for the implant receptacle and the elastic element to coincide. Thus, in these embodiments, the implant receptacle itself forms an elastic element. For example, the implant receptacle may include two leaf springs extending substantially along a common longitudinal axis, but spaced apart by a greater distance in a central region than in either of their end regions.

The shaft of the implant receptacle may be sized to receive only a single implant. In many cases, however, it is useful if the shaft is dimensioned such that multiple implants can be received, for example by stacking them on top of each other.

Preferably, the elastic element is designed as a loss prevention device. In other words, it is designed to prevent the implant from inadvertently falling out of the carrier, for example by means of a clamp described in further detail below.

Preferably, the elastic element extends substantially along the longitudinal axis of the implant receptacle and is elastic at least perpendicular to this direction. In this regard, the elastic element is arranged to be elastically deformed upon removal of the surgical implant. This embodiment causes the surgical implant to be clamped in said perpendicular direction, but does not prevent its removal in the direction of the longitudinal axis. After removal of the surgical implant, the deformation of the elastic element is canceled again.

With further advantage, the implant receptacle extends along the longitudinal axis in the direction of the first portion beyond the elastic element. This allows the wearer to be easily grasped by the implant receptacle, and the implant can be easily guided or placed onto the implant receptacle.

It is further preferred if the implant receptacle has a rounded end on the side of the first portion for receiving the implant. This makes it easier to place the implant on the implant receptacle. In addition, damage to the implant and injury to the surgical staff can be prevented.

If the carrier is inserted in a container that can be closed or sealed with a lid, it is advantageous if the first portion of the implant receptacle closes with the lid at least almost, in particular completely. This can provide additional protection against loss of the stored implant.

It is also advantageous if the elastic element is deformed in an end region in the direction of the first portion, preferably curved in the direction of the implant receptacle, in such a way that the implant is secured against accidental loss. Preferably, said end region of the implant receptacle holds the implant.

Preferably, the elastic element is shaped at its end in the direction of the first portion, preferably curved away from the implant receptacle, such that the implant elastically deforms the elastic element away from the implant receptacle during insertion. The elastic element is thus automatically deformed when the implant is inserted, without any further manual intervention being required for this purpose.

It is also convenient if the carrier has only a single elastic element. This results in a structurally simple embodiment. Of course, it is also conceivable, and within the scope of the invention, that the carrier has two or more elastic elements with the above-mentioned properties.

With further advantage, the carrier additionally has a stopper with a stop for the elastic element, which is shaped and arranged in such a way that plastic deformation of the elastic element, in particular overbending, is prevented. For example, the stopper can be formed as a sheet, in particular a bent sheet, or as a further implant receptacle. In addition, the stopper can serve as a protection of the carrier.

In this context, it is advantageous if the stopper is attached to the second portion of the implant receptacle, preferably by means of a thread. The advantage of a thread is that the stopper can simultaneously serve as a nut for fastening the carrier to a bottom of a container. As an alternative to a thread, however, the fastening can also be effected by a snap-in connection (snap-in), a press connection (press-in), an adhesive connection (glue-in) or a welded connection. The thread can be an M3 thread, for example. Furthermore, the stopper can have an opening for the elastic element so that the elastic element can be passed through the stopper. Furthermore, it is possible that the stopper is formed integrally with the elastic element. For example, the stopper and the elastic element may be formed by different portions of a one-piece bent sheet.

It is further advantageous if the first attachment portion of the implant receptacle has a conical shape. In this way, play between the implant receptacle and an opening in a bottom of a container can be prevented. This can also be realized via narrow tolerances. However, a conical shape of the first attachment portion has the advantage of more precise positionability.

Furthermore, it is advantageous if the implant receptacle in the second portion has a thread and a cross-sectional thickening, preferably a head. Thus, a bottom of the container can be clamped between the cross-sectional thickening and the stopper. Expediently, the tapered first attachment portion is thereby disposed along a longitudinal axis of the implant receptacle between the thread and the cross-sectional thickening. Thus, the cross-sectional thickening may be disposed below the retaining portion of the container and the thread may be disposed above it.

In some embodiments, the resilient member includes a second attachment portion having a recess through which the implant receptacle is at least partially passed or passable so that the resilient member is attachable to the implant receptacle. This results in a compact design.

It is also preferred if the elastic element comprises a second attachment portion which is clampable between the above-mentioned stopper and a cross-sectional thickening, preferably a head, of the implant receptacle. This embodiment allows clamping between the head and the stopper.

With further advantage, the elastic member comprises a second attachment portion having at least one first projection, preferably at least two, more preferably exactly two first projections, by means of which the elastic member is alignable with the retaining portion of the container. The alignment by such a projection is structurally particularly simple. Alternatively, of course, it is also conceivable and within the scope of the invention that the retaining section has projections with which the elastic element can be aligned on the retaining section of the container.

It is also preferred that the elastic member has a second attachment portion which has at least one second projection, preferably at least two, more preferably exactly two second projections, by means of which the stopper can be aligned on the elastic member.

Preferably, the first and/or the second extensions extend along the longitudinal axis of the implant receptacle. The first extensions and the second extensions may extend in opposite directions along the longitudinal axis of the implant receptacle. Preferably, the extensions are in the form of wings, which again is a simple design. Also preferably, the extensions extend along the longitudinal axis of the implant receptacle. Alternatively, the extensions may be arranged on the aforementioned stopper.

The implant receptacle may, for example, be made of stainless steel, in particular Inox A2, and may be manufactured as a turned part. The elastic element may, for example, comprise a spring steel and be manufactured as a stamped part and/or a bent part. In advantageous embodiments, the elastic element is formed in one piece. The stopper may also comprise, for example, stainless steel, in particular Inox A2, and may be manufactured as a stamped part and/or a bent part.

A second aspect of the invention relates to a set comprising a carrier as described above and a surgical implant, in particular an implant cooperating with the carrier in the manner described above. The implant includes a recess (for example, a recess of a bone plate) in which the implant receptacle is receivable and/or through which the implant receptacle is passable to retain the implant. The combination of the carrier with the implant results in the advantages already explained above.

Preferably, the recess is at least partially round. The recess may be wholly or partially curved or circular. It may have the shape of the implant receptacle on at least one section. The cross-section of the implant may be geometrically similar or even congruent to the cross-section of the recess. These measures facilitate the positioning of the implant at the implant receptacle.

In a third aspect, the invention relates to a set comprising a carrier as described above and a locking element for the carrier, the locking element having a body with a receiving opening through which the shaft of the implant receptacle can be passed. The locking element acts as a placeholder and prevents one or more bone plates from being inserted/received at a position where placement is not desired.

Preferably, the body has an indentation for the elastic element. Alternatively, the locking element may be at least partially elastic. Both measures ensure that the locking element can be secured to the implant receptacle.

A fourth aspect of the invention comprises a container for surgical implants, in particular bone plates. The container includes at least one base having a retaining portion, wherein a carrier as described above and/or a set as described above is attached to the retaining portion, preferably directly to the retaining portion.

The advantages of such a container result from the above explanations regarding the carrier and the set.

Provided that the container comprises a carrier according to the first aspect of the invention, the shaft of the implant receptacle of the carrier preferably extends substantially perpendicular to the bottom. In this way, multiple carriers can be arranged side by side in a particularly space-saving manner.

With further advantage, the retaining section additionally has at least one alignment recess for the first projections of the carrier described above. This creates a transition fit, which is advantageous for adjusting the holding force by the distance between the contact surfaces of the alignment recess on the one hand and the contact surfaces of the extensions on the other; for the closer said contact surfaces are to each other, the more precisely the carrier can be positioned. The alignment recess can be punched or lasered, for example. It may have a plurality of regions, for example a particularly circular region for receiving the implant receptacle and at least one particularly substantially rectangular receiving region for receiving a first extension of the elastic member as described above.

In a fifth aspect, the invention also relates to a carrier for receiving at least one surgical object, in particular an implant, in particular a screw. The carrier includes a holder having at least one receptacle for the at least one surgical object, and a securing element for the at least one object. The securing element has at least one recess for removing the at least one object through the recess. The securing element is movably connected to the holder such that the securing element is movable from a first position to a second position. Thereby, in the first position of the securing element relative to the holder, the at least one object is secured, and in the second position of the securing element relative to the holder, the at least one object can be removed from the holder through the recess of the securing element along a removal direction. Depending on the position of the securing element, the object is thus removable or not. This enables particularly good securing, which is also directly visually recognizable.

Said movement of the securing element can be translatory, for example. Preferably, the movement takes place in a plane perpendicular to the removal direction.

Preferably, the carrier is inserted in a container that can be closed with a lid. In some embodiments, the securing element protrudes beyond an edge of the container in the second position, but not also in the first position. Therefore, as long as the securing element is in the second position, the lid cannot be placed on the container. This is only possible when the securing element is transferred to the first position, in which it does not protrude beyond the edge of the container.

In other embodiments, the holder is also inserted in a container which can be closed with a lid, but the securing element is or can be brought into operative connection with the lid when the lid is closed in such a way that the securing element is brought into the first position by the closing of the lid - that is, into the position in which the surgical object is secured against falling out.

Advantageously, the holder has two or three or more receptacles for surgical objects, and the securing element has recesses corresponding to the receptacles. In the first position, the surgical objects can be secured in the receptacles by the securing element, and in the second position, the respective corresponding recesses in the securing element can be removed. By positioning the securing element accordingly, it can thus be determined whether the surgical objects are secured or can be removed.

It is convenient if the receptacles are arranged along a straight line. In this way, a space-saving arrangement can be achieved.

With further preference, the carrier, in particular the securing element, has a first spring element which is designed and arranged in such a way that the securing element is held in the first position—i.e. in the position in which the surgical object is secured against falling out. For this purpose, the first spring element can exert a spring force which acts essentially parallel to the direction in which the securing element can be moved from the second position to the first position.

Preferably, the first spring element is arranged in a first end region of the securing element. Preferably, it is formed integrally with the securing element, which results in a particularly simple design.

Further advantages result if the carrier has a latching device which is designed to latch in a latching position and to fix the securing element in the second position as soon as the securing element reaches the second position. This has the effect that the securing element cannot inadvertently return to the first position and thus prevent removal of the object. Expediently, the latching device is releasable so that the securing element can be returned to the first position in which an object that has not yet been removed is secured against falling out.

The securing element may further comprise an actuating element which, when actuated, moves the securing element into the first position. In this context, the actuating element may be designed and arranged such that it is moved by the lid when a lid is fully placed on the container, in particular in the direction in which the securing element can be moved into the second position. The actuating element may be arranged in a second end region of the securing element, which is in particular integrally formed with respect to the first end region.

The second end region can be designed as a second spring element which causes the latching device to remain in the latching position when the securing element is in the second position. For this purpose, the second spring element can exert a spring force that acts transversely, in particular perpendicularly, to the direction in which the securing element can be moved from the second position to the first position.

In a particular embodiment, the latching mechanism may be implemented in that the holder of the container has at least one contoured receiving opening for receiving the second end portion of the securing element. The receiving opening may include a passageway portion. Laterally of the passageway region, a wedge-shaped recess having an inclined upper sliding surface may be provided on the upper side of the retainer and a wedge-shaped protrusion having a lower sliding surface, a vertically extending detent surface and a step formed therebetween may be provided on the lower side of the retainer. The second spring element can ensure that the latching position is assumed when the feet described further below have been moved over the step. The force of the first spring element can press the feet against the latching surface.

With further advantage, the removal direction substantially corresponds to a longitudinal axis of a surgical object held in the recess as intended. In this way, several surgical objects can be arranged in a plane perpendicular to the longitudinal axis in a space-saving manner.

Also advantageously, the securing element can be labeled, for example by laser marking. The labeling can contain, for example, information on dimensions, use, lot number, expiration date or sequence of use. This makes it easy to identify which item needs to be loaded at which position in a container when loading the container.

It is also advantageous if the securing element has at least two feet which extend from the securing element in the direction of the holder, the feet being of elastic design and at least partially forming a counter-contour to a part of the holder, so that the feet can be latched onto the holder. In this way, the securing element can be easily and securely connected to the holder.

With further advantage, the feet are displaceable from the first position to the second position along an edge of the holder when engaged. Furthermore, it is preferred if the edges for the feet are formed by elongated holder receptacles in the holder. Both measures enable a structurally simple realization of the displaceability of the securing element.

A sixth aspect of the invention is directed to a set comprising a carrier according to the fifth aspect of the invention and at least one surgical object, in particular a surgical implant, in particular a screw. The surgical object has a cross-sectional thickening, preferably a head, which preferably rests on an edge of the receptacle of the holder of the carrier. The securing element of the carrier is arranged in its first position such that the cross-sectional thickening is blocked and cannot be removed along the removal direction. This allows for controlled removal of the surgical object.

A seventh aspect of the invention relates to a container for surgical implants, in particular screws, wherein the container comprises at least one, preferably several, carriers according to the fifth aspect of the invention and/or sets according to the sixth aspect of the invention. The advantages will be apparent from the above.

Another aspect of the invention also relates to a carrier for receiving a surgical implant having a recess, in particular a bone plate having a recess. The carrier includes a shaft for receiving the surgical implant at a recess of the implant, the shaft having means at a first end for attachment to a container. Further, the carrier includes a latch element pivotally disposed at a second end of the shaft opposite the first end of the shaft. The latch element can be pivoted to a receiving position and a locking position. In the receiving position, the bone plate with the recess can be moved onto the shaft via the locking element. By pivoting the locking element into the locking position, the locking element prevents removal of the bone plate.

The invention and its advantages are explained in more detail below with reference to numerous embodiment examples and drawings. The following are shown

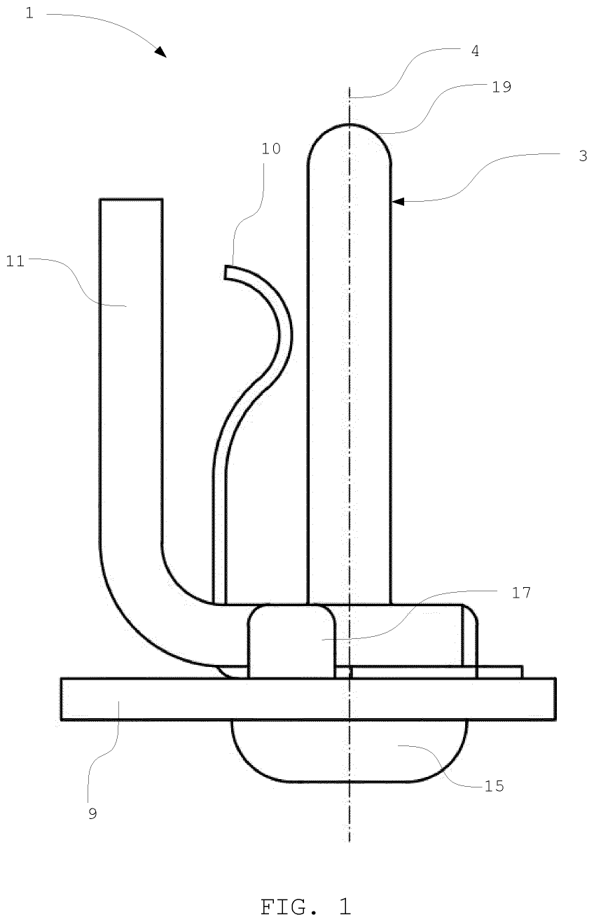

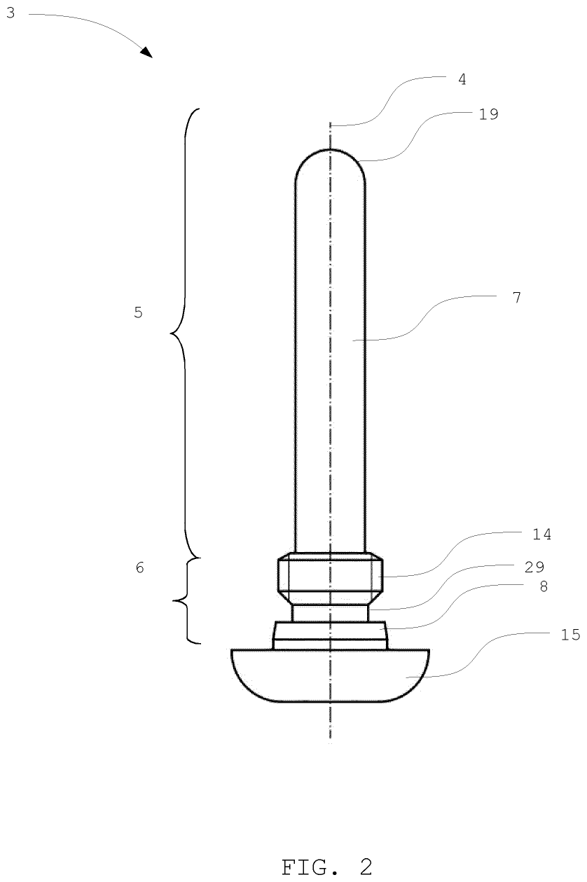

FIG. 1: a first carrier according to the invention for receiving a bone plate in a side view;

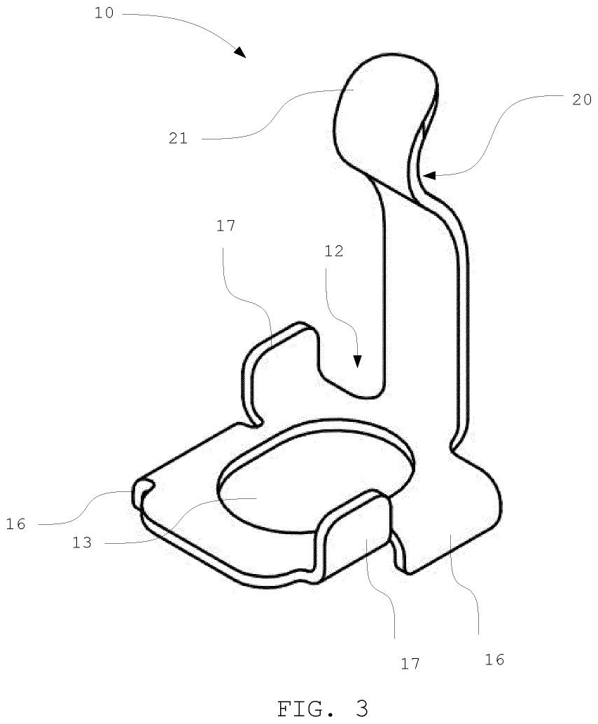

FIG. 2: a side view of an implant receptacle of the first carrier according to the invention shown in FIG. 1;

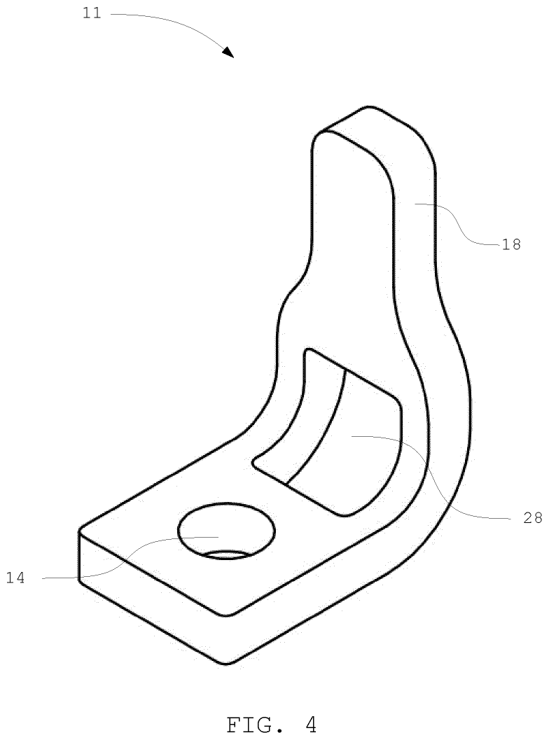

FIG. 3: a spring of the first carrier according to the invention of FIG. 1 in a perspective view;

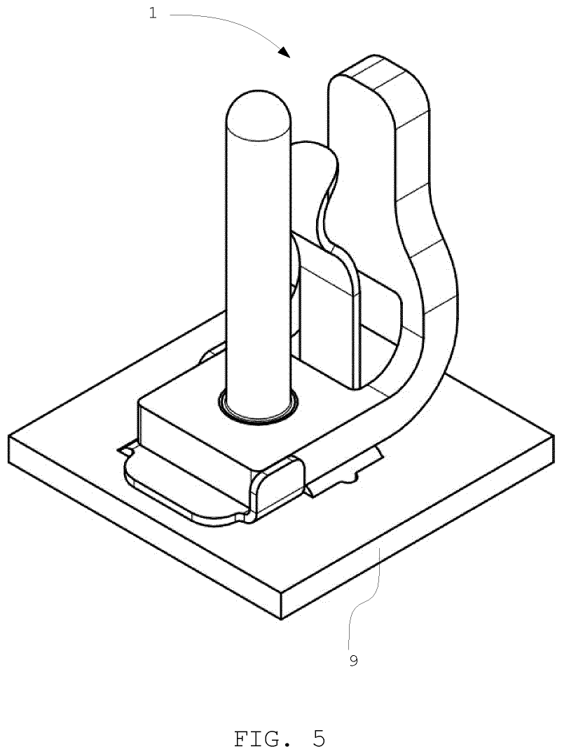

FIG. 4: a stopper of the first carrier according to the invention of FIG. 1 in a perspective view;

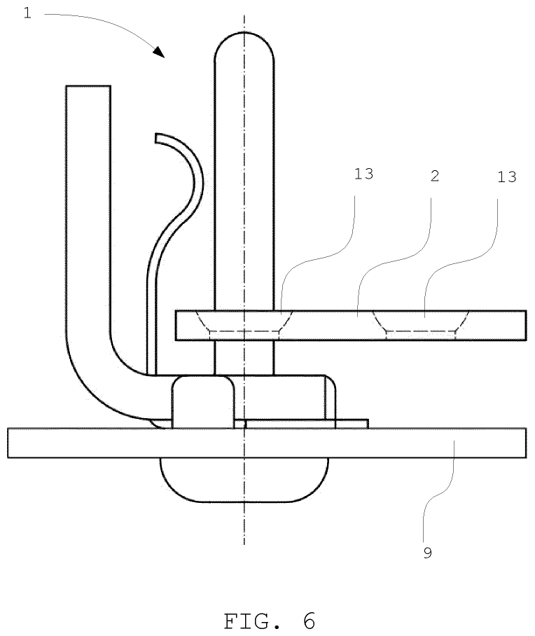

FIG. 5: a perspective view of the first carrier according to the invention attached to a holding section of a container;

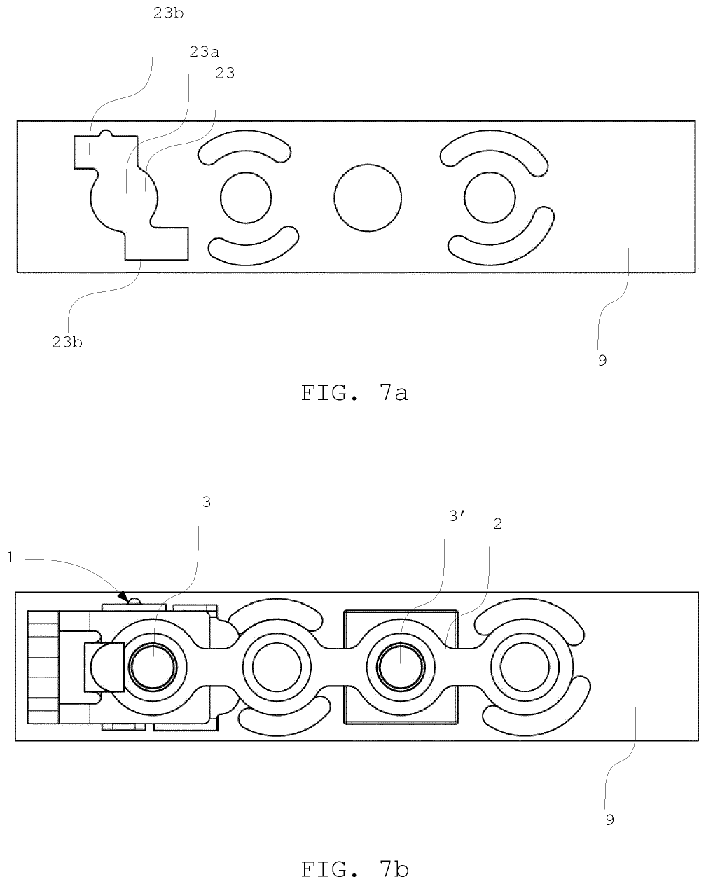

FIG. 6: a set according to the invention attached to the holding section of the container with the first carrier according to the invention and a bone plate in a lateral view;

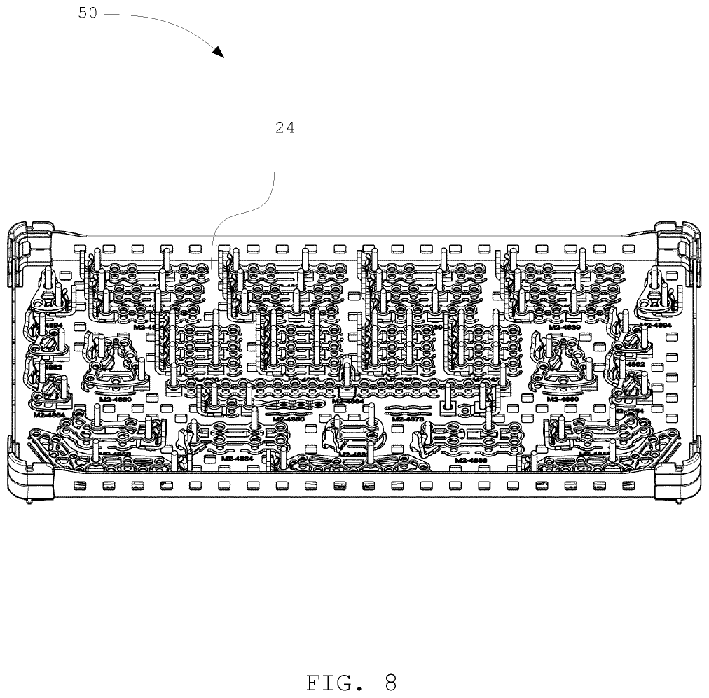

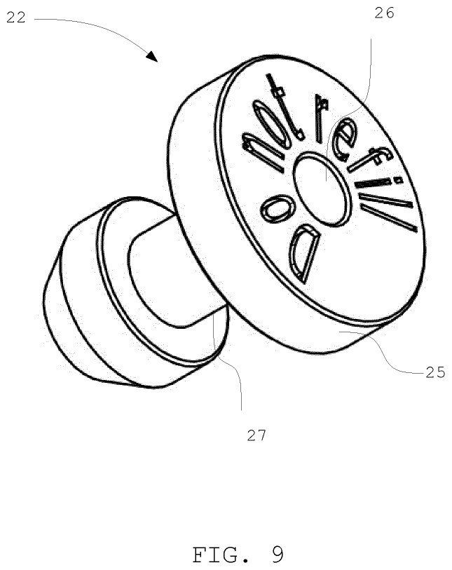

FIG. 7a: a holding section of a container in a top view;

FIG. 7b: the holding section of the container according to FIG. 7a with a set according to the invention consisting of the first carrier according to the invention and a bone plate attached thereto;

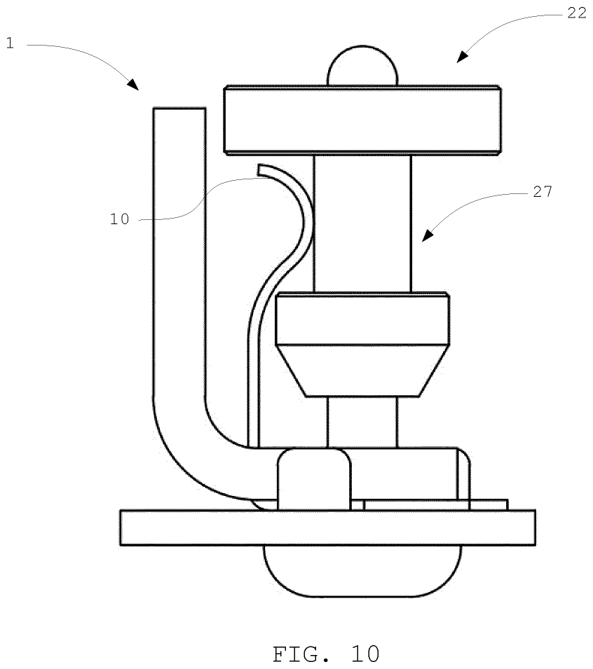

FIG. 8: a first container according to the invention with several such sets;

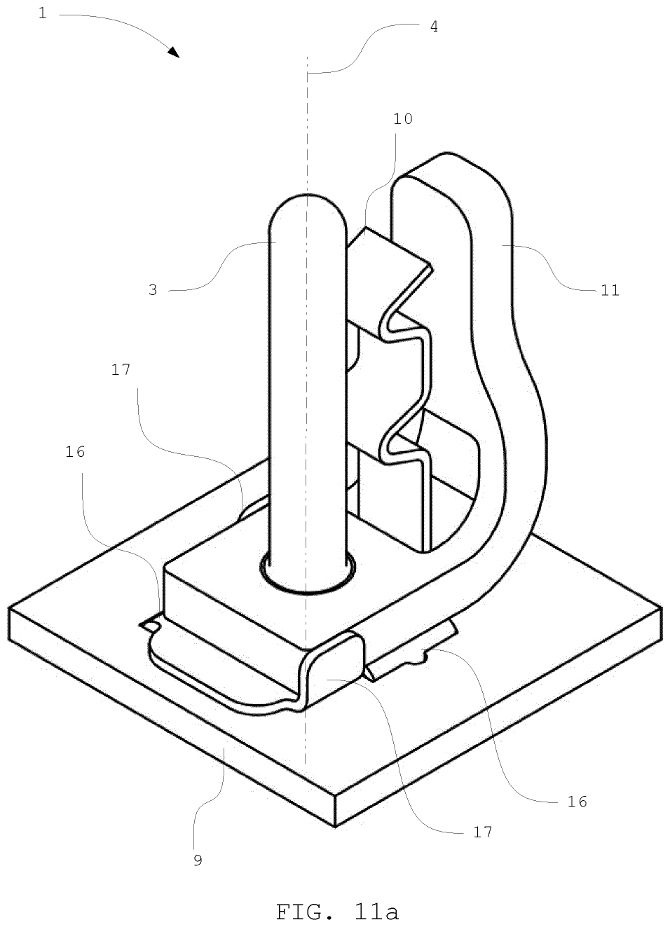

FIG. 9: a locking element for the first carrier according to the invention according to FIGS. 1 to 4 in a perspective view;

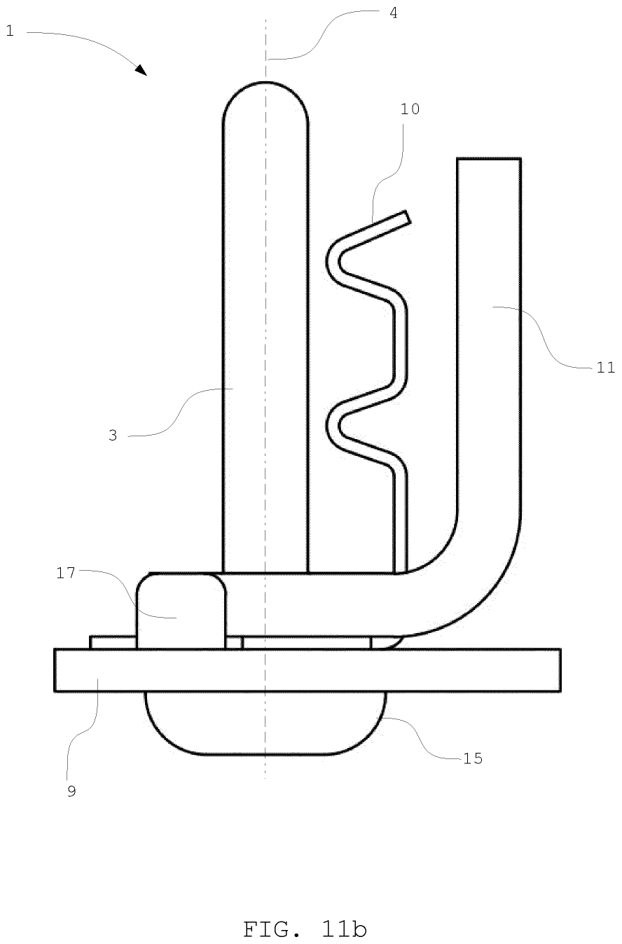

FIG. 10: the locking element according to FIG. 9 together with the carrier according to FIGS. 1 to 4 in a side view;

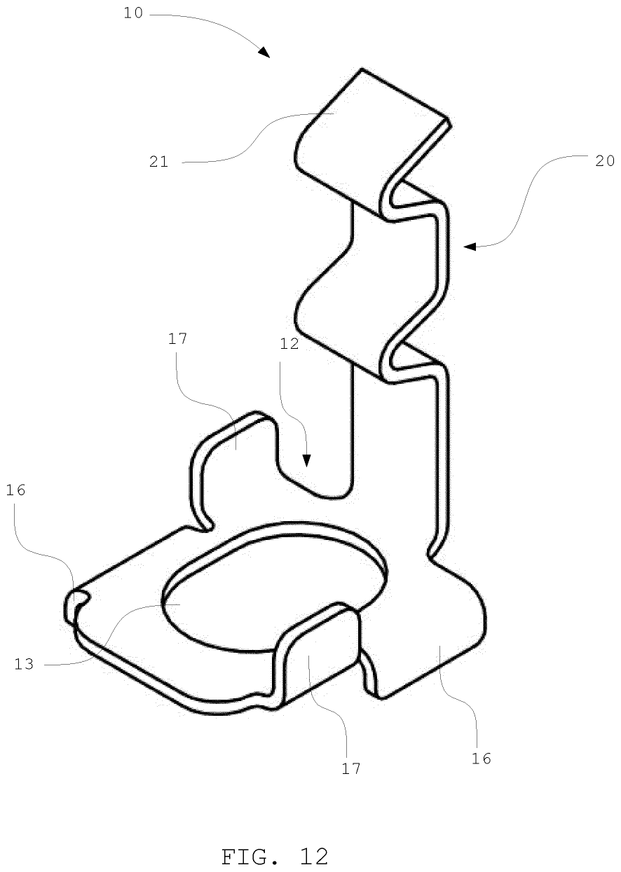

FIG. 11a: a second carrier according to the invention for receiving a bone plate in a perspective view;

FIG. 11b: the second carrier according to the invention in a side view;

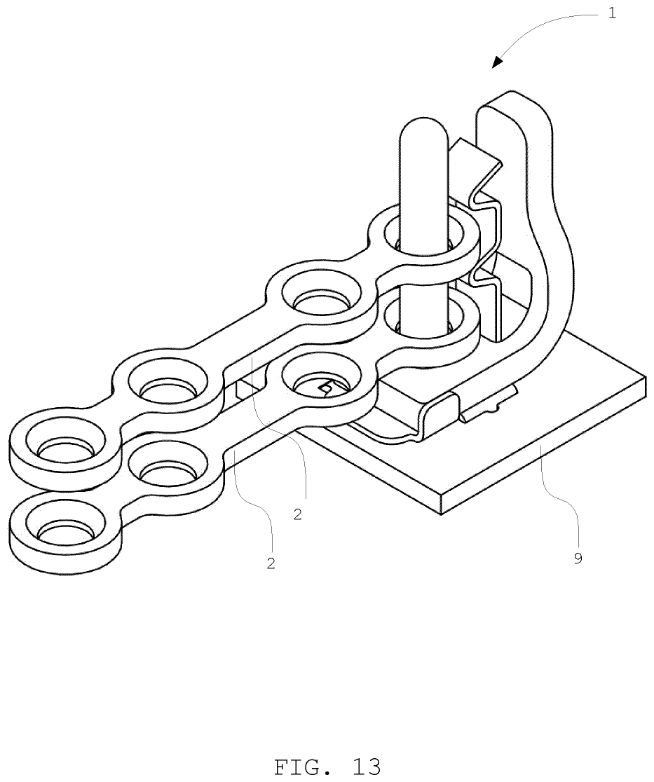

FIG. 12: a spring of the second carrier according to the invention in a perspective view;

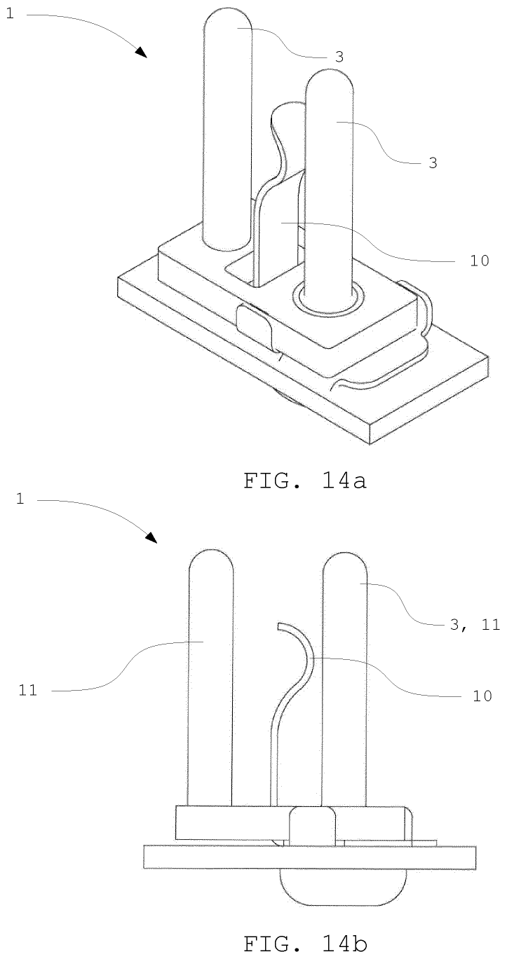

FIG. 13: the second carrier according to the invention with two bone plates in a perspective view;

FIG. 14a: a third carrier according to the invention for receiving one or two bone plates in a perspective view;

FIG. 14b: the third carrier according to the invention in a side view;

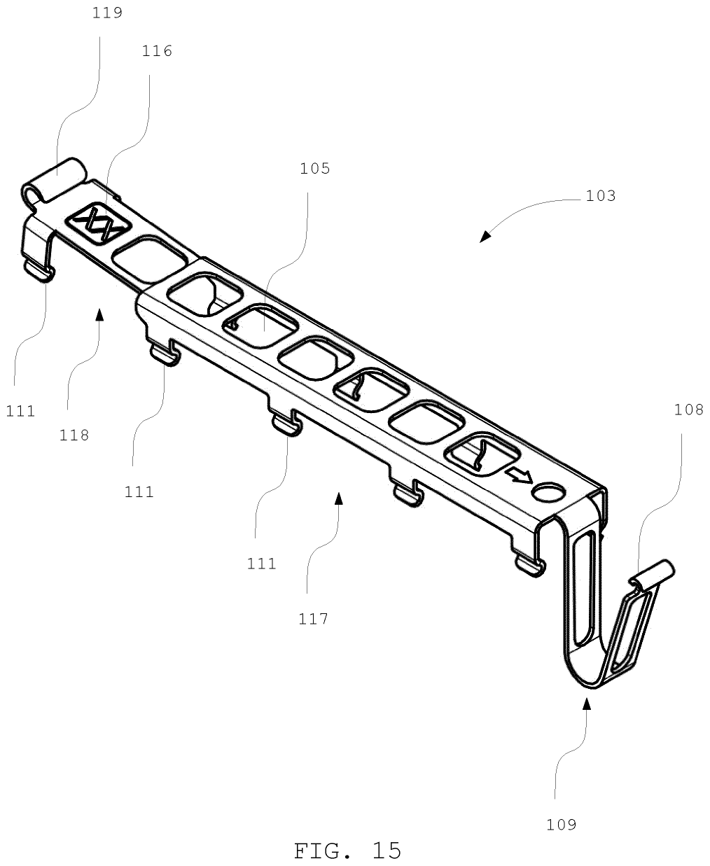

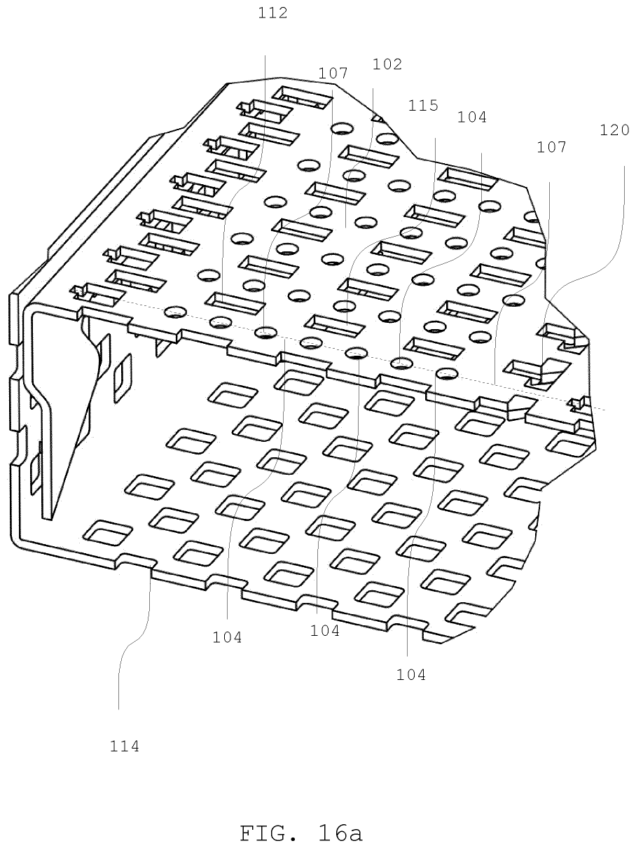

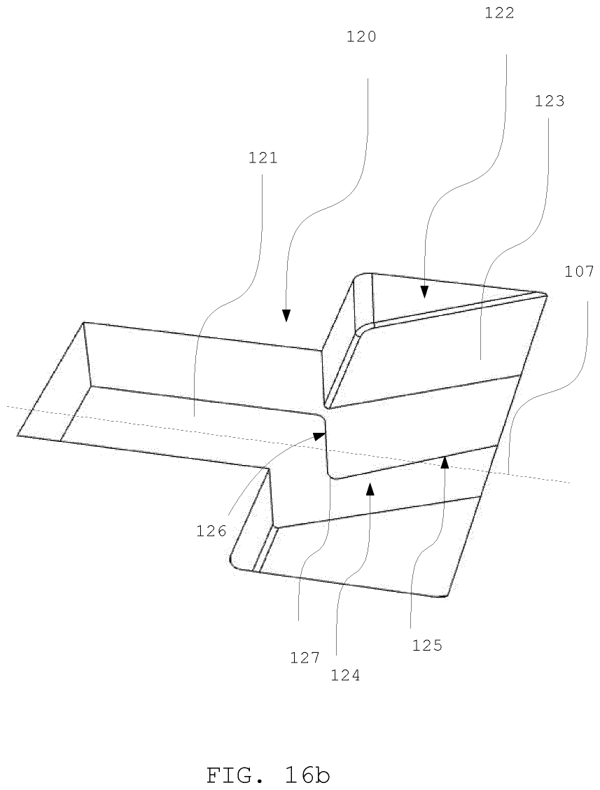

FIG. 15: a securing element of a first carrier for screws according to the invention in a perspective view;

FIG. 16a: a section of a second container according to the invention, but without a securing element, in a perspective view;

FIG. 16b: a detailed view of a contoured receiving opening of the container;

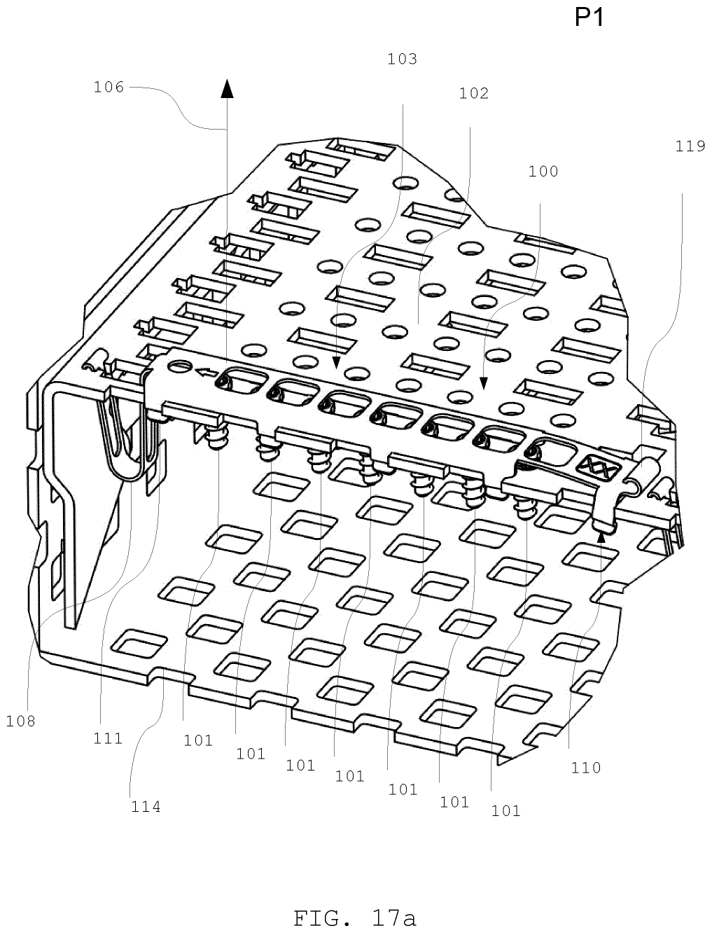

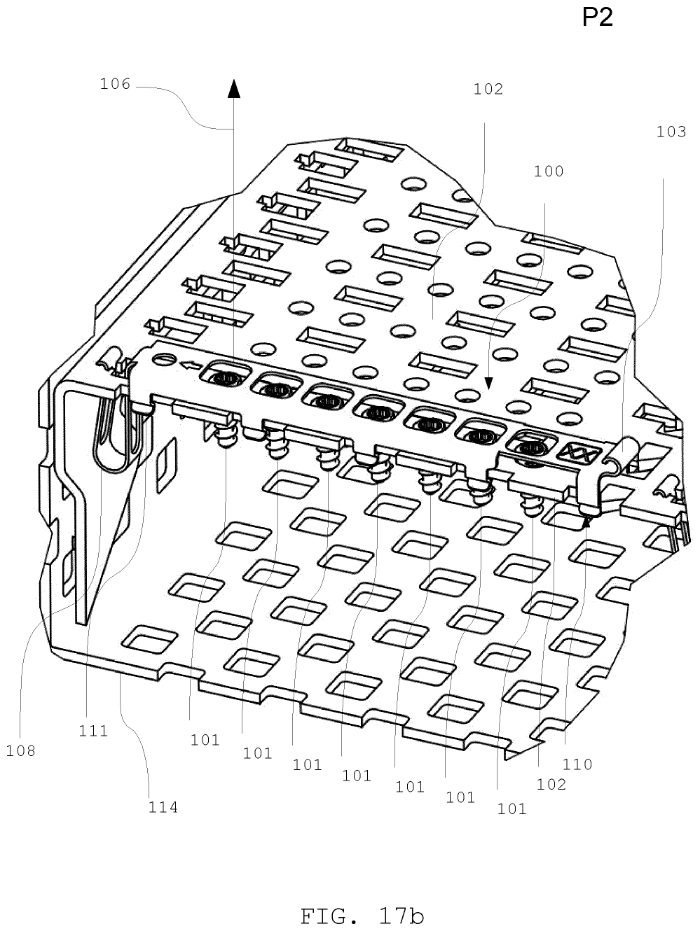

FIG. 17a: a section of the second container according to the invention with securing element in a first position, in a perspective view;

FIG. 17b: a section of the second container according to the invention with securing element in a second position, in a perspective view;

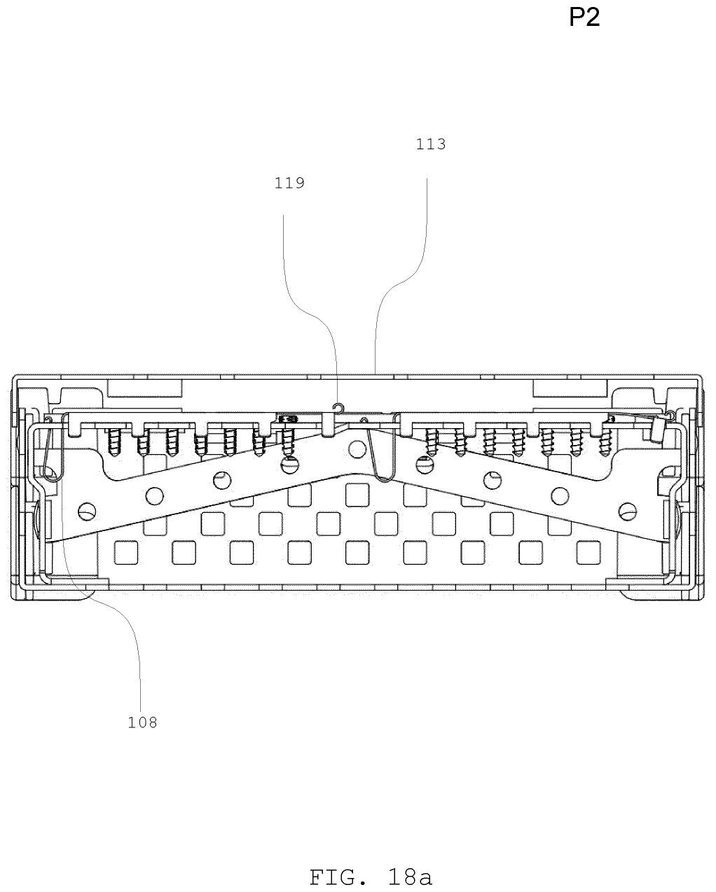

FIG. 18a: a side view of the second container according to the invention in the second position;

FIG. 18b: a side view of the second container according to the invention in the first position;

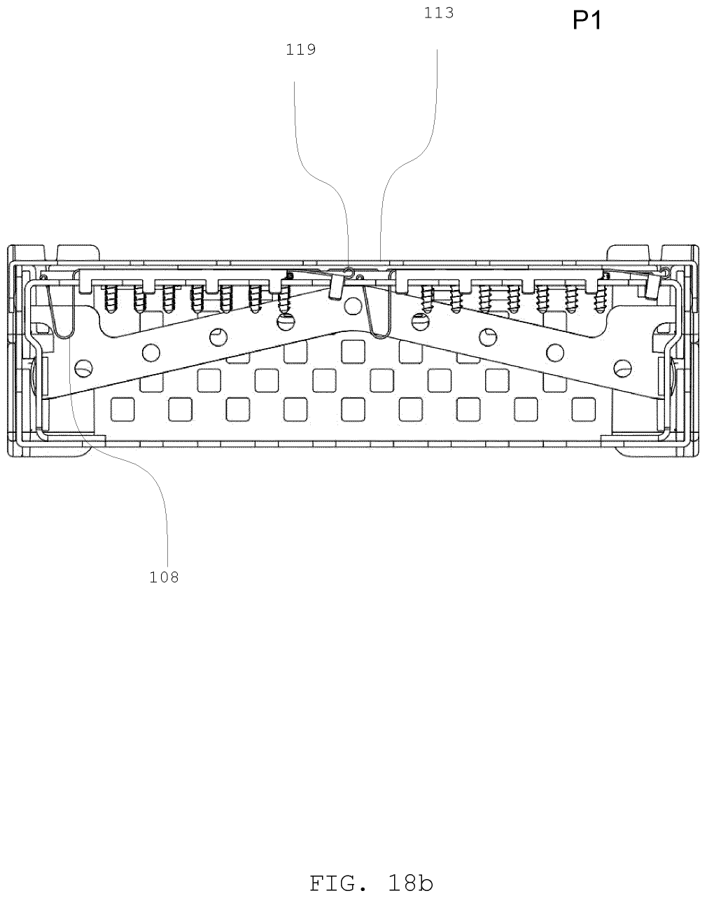

FIG. 19: a top view of the second container according to the invention;

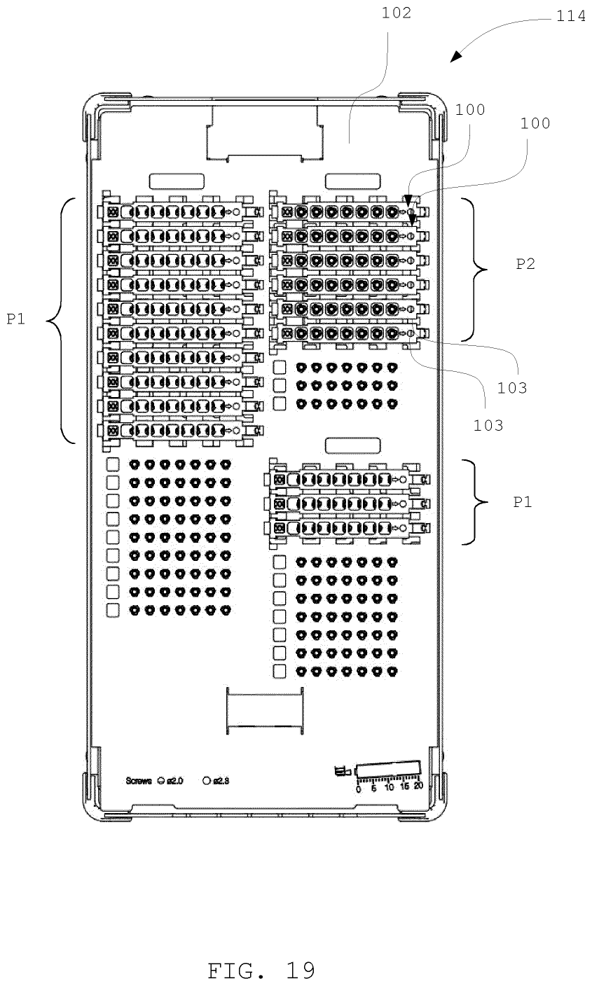

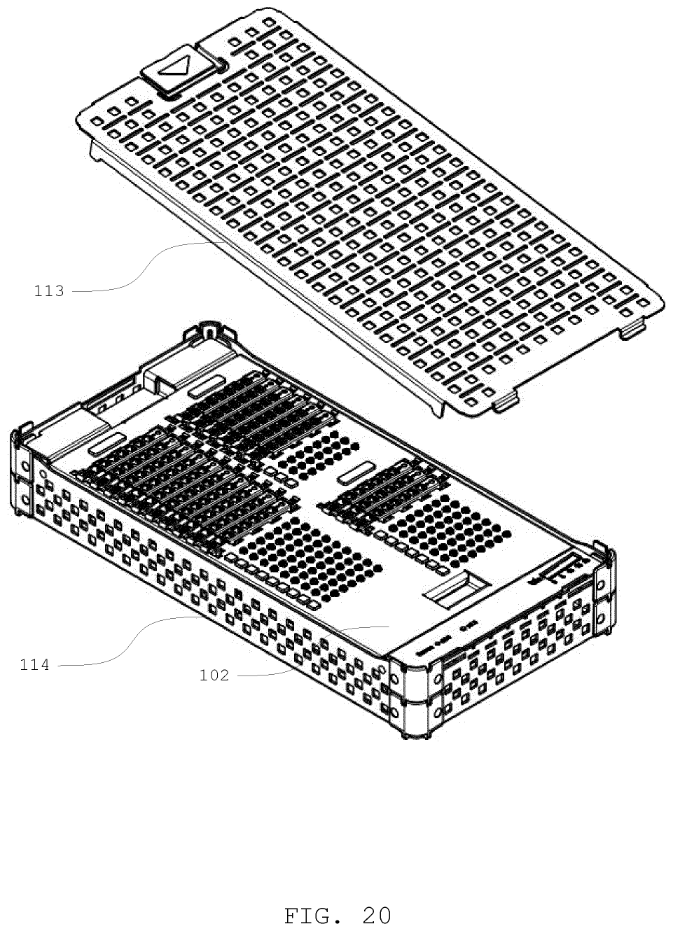

FIG. 20: a perspective view of the second container according to the invention with lid;

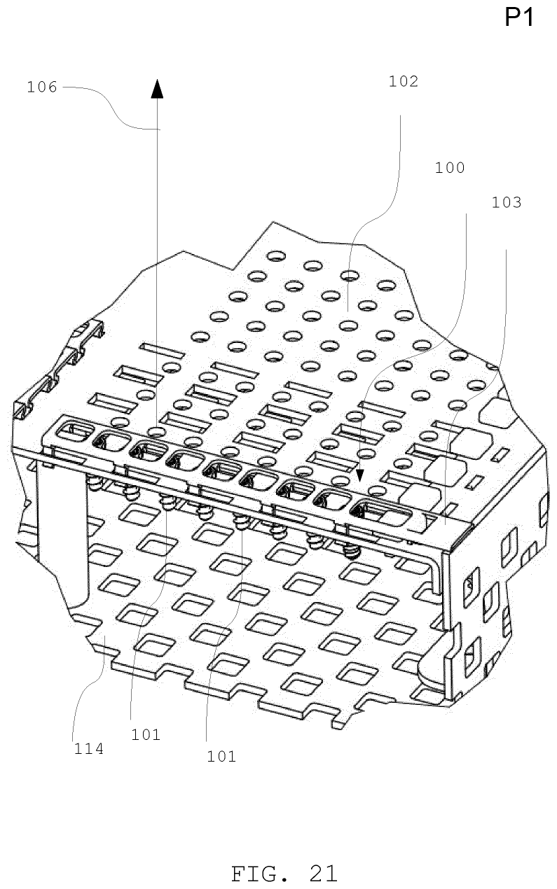

FIG. 21: a section of a third container according to the invention with securing element in a first position, in a perspective view;

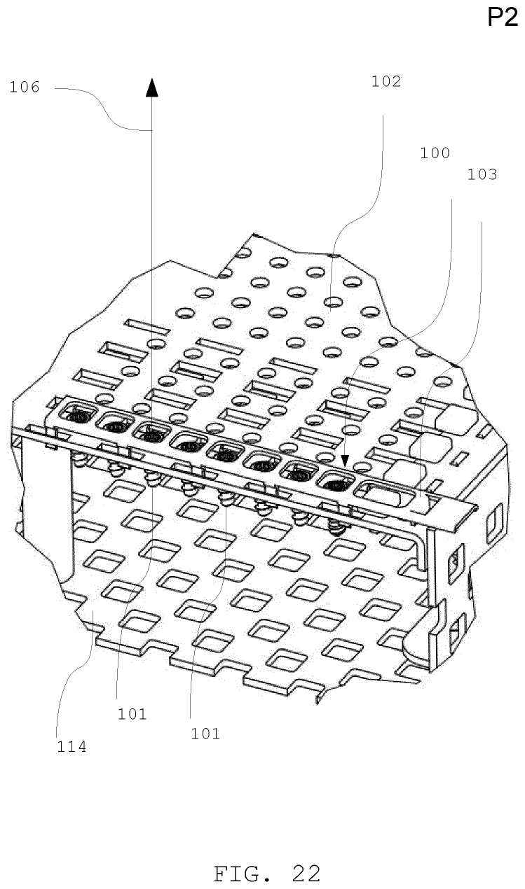

FIG. 22: a section of the third container according to the invention with securing element in a second position, in a perspective view;

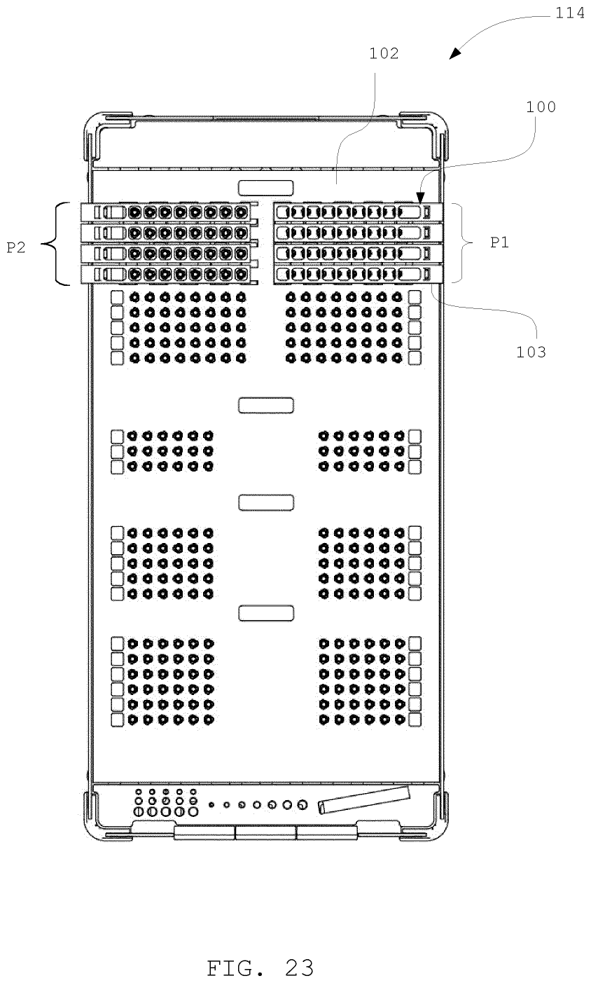

FIG. 23: the third container according to the invention in a top view;

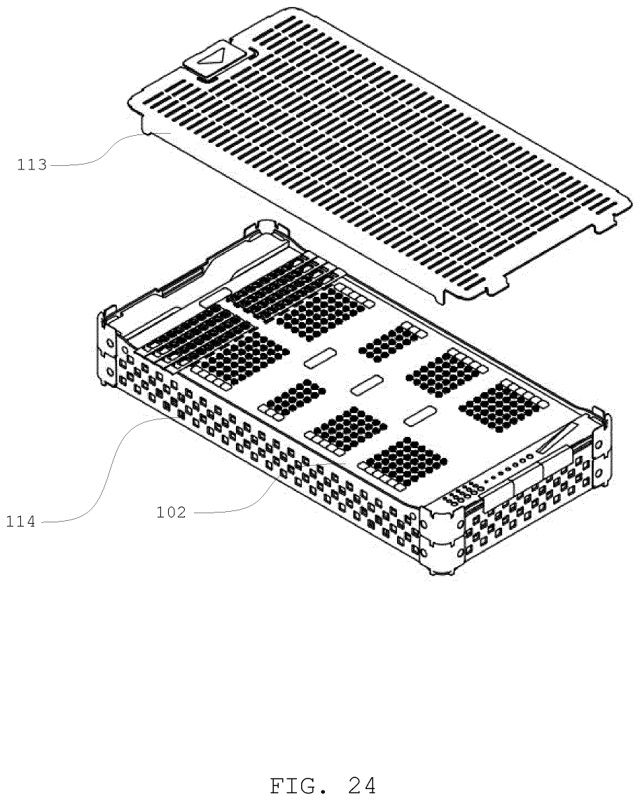

FIG. 24: the third container according to the invention with lid in a perspective view;

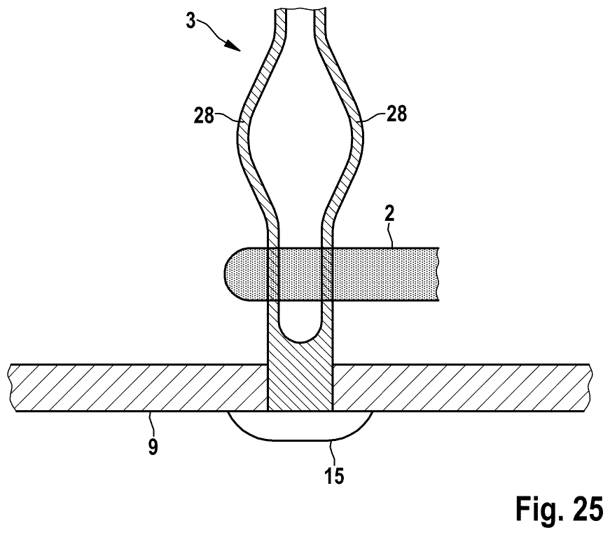

FIG. 25: a further embodiment of a carrier according to the invention for receiving a bone plate;

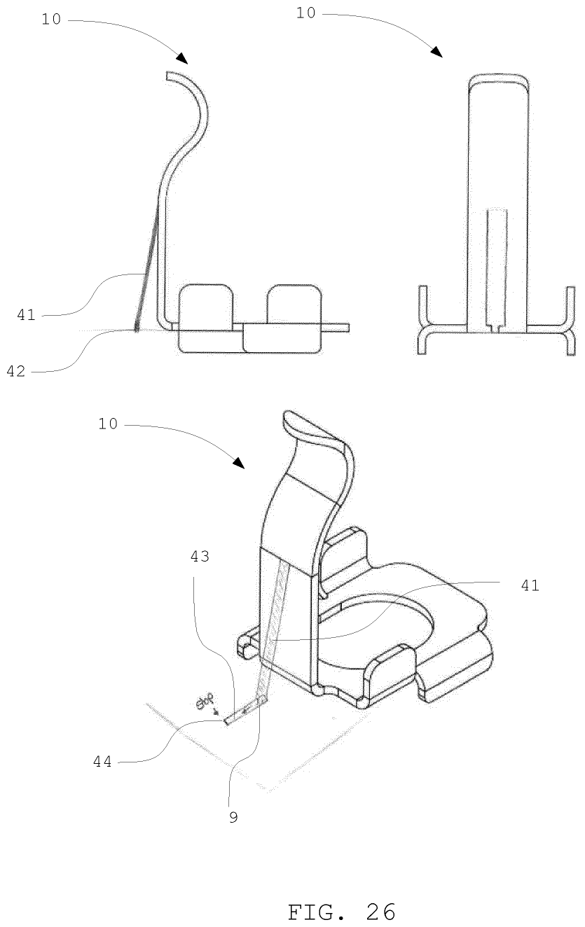

FIG. 26: a spring of a further carrier according to the invention in three views;

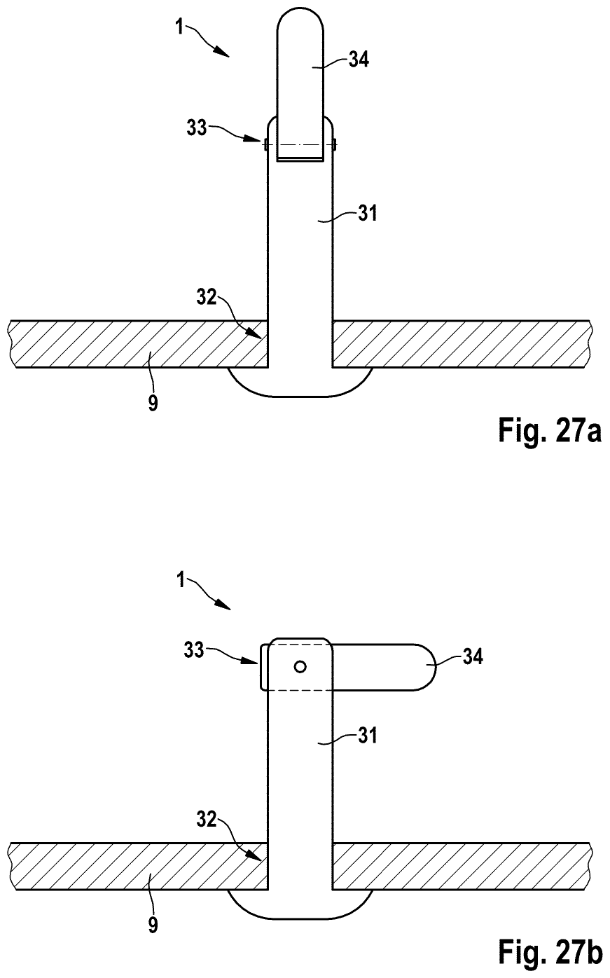

FIG. 27a: a further embodiment of a carrier attached to a base of a container with a latching element in a receiving position for a bone plate;

FIG. 27b: the carrier according to FIG. 27a with the locking element in the locking position.

FIG. 1 shows a first carrier 1 according to the invention, which is suitable for receiving an implant in the form of a bone plate not shown here (see FIG. 6). The carrier 1 contains an implant receptacle 3, an elastic element in the form of a spring 10 and a stopper 11, which are shown in detail in FIGS. 2 to 4.

According to FIG. 2, the implant receptacle 3 is substantially elongated along a longitudinal axis 4 and has a first portion 5 and a second portion 6. The first portion 5 includes a shaft 7 for receiving one or more implants at respective recesses of the implant(s). On the side of the first portion 5, the implant receptacle 3 has a rounded end 19 for receiving the implant. The second portion 6 includes a thread 14, for example an M3 thread, a first attachment portion 8 of conical shape for fastening to a holding section of a container not shown here (see FIG. 6 for this), and a cross-sectional thickening in the form of a head 15. A thread undercut 29 is located between the thread 14 and the attachment portion 8. The first attachment portion 8 is arranged along the longitudinal axis 4 of the implant receptacle 3 between the thread 14 and the cross-sectional thickening 15. As shown in FIG. 1, the stopper 11 is attached to the second portion 6 of the implant receptacle 3 by means of the thread 14. As is also evident from FIG. 1, the implant receptacle 3 extends along the longitudinal axis 4 in the direction of the first portion 5 beyond the spring 10, and the second attachment section 12 of the spring 10 is clamped between the stopper 11 and the head 15 of the implant receptacle 3.

The spring 10 shown in FIG. 3 extends along the longitudinal axis 4 of the implant receptacle 3 (see FIG. 1). The spring 10 is elastic perpendicular to this direction and arranged to be elastically deformed when the implant is removed (see FIG. 6). This provides a force component perpendicular to the longitudinal axis 4, which allows an implant held on the implant receptacle 3 to be secured. The spring 10 thus serves as a loss prevention device.

In FIG. 3, it can further be seen that the spring 10 has a second attachment section 12 with a recess 13 through which the implant receptacle 3 can be passed, allowing the spring 10 to be attached to the implant receptacle 3 (see FIG. 1). The second attachment portion 12 has two first wing-shaped projections 16. With the first projections 16, the spring 10 can be aligned with a holding section of a container not shown here (see FIG. 8). In addition, the second attachment portion 12 has two second projections 17 with which the stopper 11 can be aligned with the spring 10 (see FIG. 5). The first projections 16 and the second projections 17 extend in opposite directions with respect to the longitudinal axis 4 of the implant receptacle 3. In an end region 20, the spring 10 is curved in the direction of the first portion 5 of the implant receptacle 3 in such a way that the implant is secured against accidental loss (see in this respect FIGS. 1 and 6). At one end 21, the spring 10 is bent away in the direction of the first portion 5 of the implant receptacle 3 in such a way that an implant 2 elastically deforms the spring 10 during insertion.

The stopper 11 shown individually in FIG. 4 has a stop 18 for the spring 10. The stop 18 is shaped and arranged to prevent plastic deformation of the spring 10, in particular overbending. Furthermore, the stopper 11 has an opening 28 through which the spring 10 can be passed.

FIG. 5 shows the carrier 1 attached to a holding section 9 of a container in a perspective view.

FIG. 6 shows a set according to the invention with the first carrier 1 according to the invention and a bone plate 2. This set is attached to the holding section 9 of the container.

FIGS. 7a and 7b show a section of a retaining section 9 of a container not shown as a whole here, namely in FIG. 7a without a bone plate and in FIG. 7b with a bone plate 2. The retaining section 9 has an alignment recess 23 for the first projections 16 of the spring 10. The alignment recess 23 is composed of a central, circular region 23a and two substantially rectangular receiving regions 23b connected thereto and lying opposite one another. In the circular region 23a, the implant receptacle 3 is held and centered by means of the conical attachment portion 8 shown in FIG. 2. The first extensions 16 of the spring 10 are held in the receiving regions 23b. The bone plate 2 is also held by another implant receptacle 3′, which is identical to the implant receptacle 3 and is attached to the holding section 9 in the same way as shown in FIG. 6.

In FIG. 8, a first container 50 according to the invention is shown with several such sets. As can be seen from the figure, each carrier holds a bone plate together with a respective further implant receptacle.

FIG. 9 shows an elastic locking element 22 for the carrier 1 according to FIGS. 1 to 4. The locking element 22 has a body 25 with a receiving opening 26 through which the shaft 7 of the implant receptacle 3 can be passed (see FIG. 10). The body 25 further comprises a notch 27 in which the spring 10 engages. In this way, the locking element 22 can be secured to the implant receptacle 3 (see FIG. 10). The locking element 22 prevents bone plates from being placed on the implant receptacle 3.

The aforementioned FIG. 6 shows a set consisting of a carrier 1 and a bone plate 2 with two recesses 13. The implant receptacle 3 of the carrier 1 is received in a circular area 23a of the holding section 9 and is guided through one of the recesses 13. Through this, the bone plate 2 is held.

FIG. 8, which has also already been mentioned, shows a first container 50 according to the invention for bone plates 2. The container 50 has a base 24 with a holding section, wherein a set according to FIG. 6 is attached directly to the holding section (i.e. without any components arranged in between). The shafts 7 extend perpendicular to the base 24.

FIGS. 11a and 11b show a second carrier 1 according to the invention in a perspective view and a side view. This carrier 1 differs from the one shown in FIGS. 1 to 4 essentially in the spring 10, which is shown separately in FIG. 12. In its end region 20, the spring 10 is curved in the direction of the implant receptacle 3 not only at one point, but at two points. This allows two implants to be held at a distance from each other, which also allows the implants to be cleaned and, in particular, sterilized more easily.

The second carrier 1 according to the invention as shown in FIGS. 11a and 11b is shown in FIG. 13 together with two bone plates 2 held thereon. The carrier 1 is also attached here to a holding area 9 of a container.

A further embodiment is shown in FIGS. 14a and 14b. This carrier 1 contains two implant receptacles 3 and a spring 10. As can be seen in FIG. 14b, the implant receptacle 3 shown there on the right also functions as a stopper 11 for the spring 10. This carrier 1 can accommodate a single bone plate held by one or both implant receptacles 3, or two bone plates each held by one of the two implant receptacles 3.

FIG. 15 shows a securing element 103 of another carrier according to the invention, but which is intended to receive one or more screws. The securing element 103 contains a central area 117 with several recesses 105, through which a screw can be removed (see FIG. 17b), as well as several elastically formed feet 111. In a first end area 109, the securing element 103 has a first spring element 108 formed in one piece. Opposite the first end region 109, the securing element 103 has a second end region 118, also integrally formed, which forms a second spring element and also has elastically formed feet 111. In the second end region 118, the securing element 103 is inscribed by a laser marking 116. An actuating element 119 extending away from the feet 111, i.e. upwardly, is formed in the outermost end of the second end region 118.

FIG. 16a shows a section of a container 114 according to the invention with a holder 102. The holder 102 has a plurality of receptacles 104, each for a screw. The receptacles 104 are arranged along a straight line 107. The recesses 105 of the securing element 103 correspond to the receptacles 104 of the retainer 102. Further, the retainer 102 has elongated retainer recesses 112 arranged along a line parallel to the line 107. In addition, the bracket 102 has a plurality of contoured retainer apertures 120.

FIG. 16b includes a detailed view of one of the contoured receiving apertures 120. The receiving aperture 120 includes a rectangular passageway region 121 having a longer side and a shorter side, the longer side extending parallel to the line 107. On either side of the passageway region 121, a wedge-shaped recess 122 with an inclined upper sliding surface 123 is provided on the upper side of the retainer 102, and a wedge-shaped projection 124 with a lower sliding surface 125, a vertically extending detent surface 126, and a step 127 formed therebetween is provided on the lower side of the retainer 102.

FIGS. 17a and 17b show the container 114 with the retainer 102 and a securing element 103. A screw 101 is inserted in each of the receptacles 104. The first spring element 108 is inserted in an opening in the bracket 102, and the feet 111 of the second end portion 118 are inserted in two adjacent contoured receiving openings 120. The feet 111 of the middle portion 117 extend in the direction of the bracket 102 and are engaged in the bracket recesses 112. The longitudinal extent of the feet 111 of the center portion 117 in the direction of the line 107 is less than the longitudinal extent of the retainer recesses 112 in that direction. This has the effect of movably connecting the securing element 103 to the holder 102. In this way, it can be moved, i.e. translationally moved, from a first position P1 shown in FIG. 17a to a position P2 shown in FIG. 17b. This is made possible by the fact that the feet 111 are displaceable along an edge 115 of the holder when engaged. The edges 115 are formed by the elongated bracket recesses 112 of the bracket 102.

In the first position P1 shown in FIG. 17a, the screws 101 are secured by the securing element 103. This is because, since the recesses 105 of the securing element and the receptacles 104 of the holder 102 are not aligned with each other, the screws 101 cannot be removed along a removal direction 106 that coincides with the longitudinal axis of the screw 101. This becomes possible only in the second position P2 shown in FIG. 17b. In this second position P2, the screw 101 can be removed from the receptacle 104 through the recess 105 along the removal direction 106.

The first spring element 108 is configured and arranged to hold the securing element 103 in the first position P1. FIGS. 17a and 17b also reveal a releasable latching device 110 that can latch and thus fix the securing element 103 in the second position P2, once the securing element 103 reaches the second position P2.

FIGS. 18a and 18b show in detail, in lateral views, how the movement of the securing element 103 is achieved by cooperating with a lid 113 of the container 50. In the second position P2 shown in FIG. 18a, the feet 111 of the first end portion 118 are pressed against the latching surfaces 126 by the first spring element 108. In this second position P2, the lid 113 is not yet fully attached, so that there is not yet any contact with the actuating element 119.

This contact does not occur until the lid 113 is fully fitted, as shown in FIG. 18b. The cover 113 presses down the actuating element 119. This moves the feet 111 of the second end portion 118 over the step 127, so that the securing element 103 moves from the second position P2 to the first position P1 by the spring force generated by the first spring element 108. In the process, the feet 111 of the second end portion 118 slide along the sliding surfaces 125 of the contoured receiving openings 120.

After the lid 113 is lifted off, the securing element 103 remains in the first position P1 due to the force caused by the first spring element 108. By exerting a force directed parallel to the line 107 on the actuating element 119, the feet 111 can be moved along the sliding surfaces 123, 125 up to above the step 127, whereby the second position P2 is reached and the feet 111 of the second end portion 118 move upwards and thus back to the latching position due to the resilient action of the first end portion 118.

The transfer of the securing element 103 from the second position P2 to the first position P1 can of course also be performed manually, i.e. without the action of the cover 113.

FIG. 19 shows a first container 114 according to the invention with a holder 102 and a plurality of carriers 100, each of which comprises a securing element 103. Here, some of the securing elements 103 are in the first position P1 and the remaining ones are in the second position P2.

In FIG. 20, the container 114 is shown together with a lid 113 with which it can be closed.

FIGS. 21 to 24 show a further example of an embodiment. The securing elements 103 of the carriers 100 shown in FIG. 23 on the left are in the second position P2, in which the securing elements 103 protrude beyond the edge of the container 114. The securing elements 103 of the carriers 100 shown in FIG. 23 on the right are in the first position P1 and do not protrude beyond the edge of the container 114. As long as at least one of the securing elements 103 is in the second position P2, no lid 113 can be placed on the container 114. This is only possible when the securing elements 103 are transferred to the first position P1, in which they do not protrude beyond the edge of the container 114. Thus, the screws 101 are always secured against falling out when the lid 113 is in place.

The carrier 1 according to the invention for a bone plate 2 shown in FIG. 25 contains two leaf springs 28 which extend substantially along the common longitudinal axis 4, but which are spaced apart from each other by a greater distance in a central region. When a bone plate 2 with a recess is placed over the leaf springs 28, the leaf springs 28 are pressed against each other and the bone plate 2 can be slid over the central region. After the bone plate 2 has passed the central area, the leaf springs 28 spring apart again and thus secure the bone plate 2 against accidental falling out.

FIG. 26 shows a spring 10 of a further carrier 1 according to the invention. The spring 10 has a lug 41 with an end 42 that is guided in a rail 43. One end 44 of the rail 43 forms a stop which prevents excessive bending of the spring 10.

FIGS. 27a and 27b show another carrier 1 for a bone plate. The carrier 1 includes a shaft 31 for receiving the bone plate at one of its recesses. At a first, here lower end 32, the shaft 31 has means for attachment to a retaining section 9 of a container. Further, the carrier 1 includes a locking element 34 pivotally disposed at a second, here upper, end 33 of the shaft 31 opposite the first end 32 of the shaft 31. The locking element 34 can be pivoted to a receiving position shown in FIG. 27a and to a locking position shown in FIG. 27b. In the receiving position, the bone plate with the recess can be moved over the locking element 34 onto the shaft 31. By pivoting the locking element 34 into the locking position, the locking element 34 prevents removal of the bone plate.

1-37. (canceled)

38. A carrier for receiving a surgical implant, comprising:
an implant receptacle, wherein the implant receptacle is substantially elongated along a longitudinal axis and has a first portion and a second portion,
wherein the first portion has a shaft for receiving the surgical implant at a recess of the implant,
the second portion having a first attachment portion for attachment to a retaining portion of a container, and
at least one elastic element, for securing the implant,
wherein the elastic element is constructed and arranged to secure an implant held on the implant holder by a force component essentially perpendicular to the longitudinal axis.

39. The carrier according to claim 38, wherein the elastic element is a loss prevention device.

40. The carrier according to claim 38, wherein the elastic element extends along the longitudinal axis of the implant receptacle and is elastic at least perpendicular to this direction, wherein the elastic element is arranged to be elastically deformed upon removal of the surgical implant.

41. The carrier according to claim 38, wherein the implant receptacle extends along the longitudinal axis in the direction of the first portion beyond the elastic element.

42. The carrier according to claim 38, wherein the implant receptacle has a rounded end on the side of the first portion for receiving the implant.

43. The carrier according to claim 38, wherein the elastic element is shaped in an end region in the direction of the first portion, preferably curved in the direction of the implant receptacle, in such a way that the implant is secured against accidental loss.

44. The carrier according to claim 38, wherein the elastic element is shaped at its end in the direction of the first portion, preferably curved away from the implant receptacle, in such a way that the implant elastically deforms the elastic element away from the implant receptacle during insertion.

45. The carrier according to claim 38, wherein the carrier has only one elastic element.

46. The carrier according to claim 38, wherein the support additionally comprises a stopper with a stop for the elastic element, which is shaped and arranged in such a way that a plastic deformation of the elastic element is prevented.

47. The carrier according to claim 46, wherein the stopper is fixed to the second portion of the implant receptacle, preferably by means of a thread.

48. The carrier according to claim 38, wherein the first attachment portion has a conical shape.

49. The carrier according to claim 48, wherein the implant receptacle in the second portion has a thread and a cross-sectional thickening, preferably a head, and wherein the conical first attachment portion is preferably arranged along the longitudinal axis of the implant receptacle between thread and the cross-sectional thickening.

50. The carrier according to claim 38, wherein the elastic element has a second attachment portion that defines a recess through which the implant receptacle is at least partially led through or can be led through, so that the elastic element can be attached to the implant receptacle.

51. The carrier according to claim 44, wherein the elastic element has a second attachment portion which can be clamped between the stopper and a cross-sectional thickening of the implant receptacle.

52. The carrier according to claim 38, wherein the elastic element has a second attachment portion which has at least one first extension, preferably at least two, more preferably exactly two extensions, with which the elastic element can be aligned on the holding section of the container.

53. The carrier according to claim 46, wherein the elastic element has a second attachment portion, which has at least one second extension, preferably at least two, particularly preferably exactly two second extensions, with which the stopper can be aligned on the elastic element.

54. The carrier for receiving at least one surgical object, comprising:
a holder with at least one receptacle for the at least one surgical object,
a securing element for the at least one object,
wherein
the securing element defines at least one recess for removal of the at least one object through the recess,
the securing element is movably connected to the holding device, so that the securing element can be moved from a first position to a second position,
in the first position of the securing element relative to the holder, the at least one object is secured, and
in the second position of the securing element relative to the holder, the at least one object can be removed from the holder through the recess of the securing element along a removal direction.

55. The carrier according to claim 54, wherein the carrier is inserted into a container which is closable by a lid, and wherein the securing element is or can be brought into operative connection with the lid when the lid is closed in such a way that the securing element is brought into the first position by the closing of the lid.

56. The carrier according to claim 54, wherein the holder has two or three or more receptacles for surgical objects and wherein the securing element has recesses corresponding to the receptacles, wherein the surgical objects can be secured in the first position in the receptacles by the securing element and can be removed in the second position through the respective corresponding recesses in the securing element.

57. The carrier according to claim 56, wherein the receptacles are arranged along a straight line.

58. The carrier according to claim 54, wherein the carrier comprises a first spring element which is formed and arranged in such a way that the securing element is held in the first position.

59. The carrier according to claim 58, wherein the first spring element is arranged in a first end region of the securing element and is preferably formed integrally with the securing element.

60. The carrier according to claim 54, wherein the carrier has a latching device which is designed to latch and fix the securing element in the second position as soon as the securing element reaches the second position.

61. The carrier according to claim 60, wherein the latching device is releasable.

62. The carrier according to claim 54, wherein the removal direction substantially corresponds to a longitudinal axis of an object held in the recess as intended.

63. The carrier according to claim 54, wherein the securing element is labeled.

64. The carrier according to claim 54, wherein the securing element has at least two feet extending from the securing element in the direction of the holder, wherein the feet are of elastic design and wherein the feet at least partially form a counter contour to a part of the holder, so that the feet can be latched onto the holder.

65. The carrier according to claim 64, wherein the feet in the engaged state are displaceable along an edge of the holding frame from the first position to the second position.

66. The carrier according to claim 65, wherein the edges for the feet are formed by elongated holder recesses in the holder.
